SEQUENCE LISTING (6 sequences)

SEQ ID NO: 1 - DNA, 29 bp, Homo sapiens
  primer_bind (1)..(29)
gctggactgttagatgataacggaggtac

SEQ ID NO: 2 - DNA, 28 bp, Homo sapiens
  primer_bind (1)..(28)
tgtcagcatcccagagtggcaataggag

SEQ ID NO: 3 - DNA, 24 bp, Homo sapiens
  primer_bind (1)..(24)
ctacaagttctggcttagacatgg

SEQ ID NO: 4 - DNA, 24 bp, Homo sapiens
  primer_bind (1)..(24)
cgattttcagagtcgactgtccag

SEQ ID NO: 5 - DNA, 20 bp, Homo sapiens
  primer_bind (1)..(20)
cgattttcagagtcgactgt

SEQ ID NO: 6 - DNA, 21 bp, Homo sapiens
  primer_bind (1)..(21)
cgacggtgagaggcaccctcc

(19) United States
(12) Patent Application Publication
(10) Pub. No.: US 2006/0160119 A1
(43) Pub. Date: Jul. 20, 2006

(54) Genetic screening for improving treatment of patients diagnosed with depression

(76) Inventors: Barbara Turner, Johnson City, TN (US); Barney Miller, Johnson City, TN (US)

Correspondence Address: DONNA J. RUSSELL, 1492 ANTHONY WAY, MT. JULIET, TN, 37122, US

(21) Appl. No.: 11/331,459

(22) Filed: Jan. 14, 2006

Related U.S. Application Data
(60) Provisional application No. 60/643,830, filed on Jan. 14, 2005.

(51) Int. Cl. C12Q 1/68; A61K 31/137

(52) U.S. Cl. 435/6; 514/649

(57) ABSTRACT

Disclosed is a method for screening patients to determine whether or not SSRI therapy is likely to alleviate symptoms of depression in those patients. The method provides a polymorphism at position −1019 of the 5-HT1A gene that is predictive of likelihood of improvement of symptoms and a polymorphism at position 102 of the 5-HT2A gene that is predictive of likelihood of unwanted side effects related to SSRI therapy administered to a patient.

### CROSS-REFERENCE TO RELATED APPLICATIONS

This application claims the benefit of priority of earlier-filed U.S. provisional application No. 60/643,830.

### FIELD OF THE INVENTION

The invention relates to methods for screening individuals to identify those who will more likely benefit from certain pharmaceutical therapies for depression. More specifically, the invention relates to a method for distinguishing between individuals who will respond and those who will not likely respond to serotonin reuptake inhibitors by experiencing favorable physiological results and decreased side effects.

### BACKGROUND OF THE INVENTION

Worldwide, depression is one of the leading causes of disability. Each year, 9.5% of the population experience depressive illness (about 18.8 million American adults). Major depression affects 16 million people in the United States each year. Several types of antidepressant medications are used to treat depressive disorders, including monoamine oxidase inhibitors (MAOIs), tricyclic antidepressants, and newer medications such as the selective serotonin reuptake inhibitors (SSRIs). A deficit in serotonin (5-hydroxy-tryptamine or 5-HT) activity has been proposed to be either a major cause of depression or an important factor in predisposing an individual to depression, since disorders in serotonergic activity can contribute to many of the symptoms of major depression (e.g., altered mood, appetite, sleep activity, sexual function, cognitive function, and increased tendency toward suicide). Existing antidepressant drugs primarily influence the functioning of either or both of two neurotransmitters in the brain—serotonin and norepinephrine. Older medications—tricyclic antidepressants (TCAs) and monoamine oxidase inhibitors (MAOIs)—affect the activity of both of these neurotransmitters simultaneously. SSRIs, however, generally have fewer side effects than either tricyclics or MAOIs. Combinations of these medications are also prescribed for many patients. Antidepressant medications must be taken regularly for 3 to 4 weeks (and as many as 8 weeks, in some cases) before the full therapeutic effect occurs, although some patients may experience improvement very soon after initiation of therapy.

Not all patients who are diagnosed with depression experience favorable outcomes following administration of either paroxetine or other SSRIs. In some, the symptoms of depression persist. Some experience side effects such as nausea, increased infection, diarrhea, constipation, decreased appetite, sleepiness, dizziness, sweating, abnormal vision, and/or sexual side effects. Of even more concern are the possible effects such as increased anxiety, agitation, panic, irritability, hostility, aggression, and/or sleeplessness that are experienced by some individuals who take these medications. Since depression may predispose certain individuals to consider suicide, identifying effective medication and minimizing side effects so that patients adhere to the treatment regimen is even more important.

In the past 20 years, the United States Food and Drug Administration has approved a variety of new antidepressants, including SSRIs. With these therapeutics, approximately 80-90% of all depression can be ultimately treated effectively, once an appropriate medication is identified. Identification of effective agents is key to effective therapy, but, when dose adjustments and trials with multiple agents are taken into account, it can often take 6 months to 1 year for a patient to experience effective relief. For some patients, the process of finding an appropriate pharmaceutical therapy takes well over a year. Up to 30-45 percent of depressed patients do not receive adequate relief with the first drug prescribed, and up to 30 percent of patients placed on antidepressant therapy discontinue it within the first month due to inadequate response, side-effects, or both.

What is needed is a method for distinguishing between patients that are likely to benefit from SSRI therapy and those that are more likely to experience unwanted side-effects and discontinue the prescribed medication.

### SUMMARY OF THE INVENTION

The present invention relates to a method for identifying individuals who are more or less likely to elicit a favorable physiological response to standard SSRI antidepressant therapy. In one embodiment, the invention provides a genetic test to detect a polymorphism associated with decreased effectiveness of SSRI antidepressant therapy. In the method of the invention, individuals who are less likely to benefit from standard single-medication SSRI therapy are identified by the presence of the G/G allelic genotype at position −1019 of the serotonin 1A receptor (5-HT_{1A}R) promoter. Individuals who will be more likely to benefit from SSRI monotherapy are identified by the non-G/G genotype whereas individuals more likely to benefit from non-SSRI therapy to target noradrenergic and/or dopaminergic systems are identified by the G/G genotype.

The invention also provides a method for identifying individuals who are more or less likely to experience unwanted side effects of SSRI therapy, the method comprising detecting the presence or absence of the C/C genotype at position 102 in the serotonin 2A receptor (5HT_{2A}R).

In one embodiment of a method for screening a patient to determine whether or not that patient is likely to benefit from therapeutic administration of a selective serotonin reuptake inhibitor for treatment of depression, the method comprises identifying the genotype of the individual at position −1019 in the DNA sequence of the 5-HT1A receptor gene, with the presence of the G/G genotype indicating that selective serotonin reuptake inhibitor treatment will be less likely to result in successful treatment of symptoms and the presence of a non-G/G genotype at the same position indicating that SSRI treatment will be more likely to result in successful treatment of symptoms.

In one embodiment of a method for screening individuals who are more likely to experience unwanted side-effects related to administration of a selective serotonin reuptake inhibitor for the treatment of depression, the method comprises identifying the genotype of the individual at position 102 in the DNA sequence of the 5-HT2A receptor gene, with the presence of the C/C genotype indicating that selective serotonin reuptake inhibitor treatment will be more likely to result in unwanted side-effects and the presence of a non-C/C genotype at the same position indicating that SSRI treatment will be less likely to result in unwanted side-effects. In one embodiment, the selective serotonin reuptake inhibitor may be paroxetine.

The invention also provides a kit or kits for pre-screening one or more patients prior to choosing a specific antidepressant therapy. In one embodiment, the kit comprises a primer pair comprising SEQ ID NO: 1 and SEQ ID NO: 2. A kit may also comprise reagents for isolating DNA from the one or more patients. A kit may also comprise reagents for amplification of DNA from the one or more patients.

A kit may also comprise SEQ ID NO: 3, SEQ ID NO: 4, and SEQ ID NO: 5, or SEQ ID NO: 3 and SEQ ID NO: 6, and a kit as described by the invention may also comprise primer pairs for identifying the polymorphism at −1019 of 5-HT1A (e.g., SEQ ID NO: 1 and SEQ ID NO: 2) and primer pairs for identifying the polymorphism at 102 of 5-HT2A (SEQ ID NO: 3 with SEQ ID NO: 4 and SEQ ID NO: 5, or with SEQ ID NO: 6), for example.

### BRIEF DESCRIPTION OF THE DRAWINGS

FIG. 1 is a bar graph depicting the relationship between 5-HT1A receptor genotype and drug class. Patients taking either an SSRI monotherapy or a non-SSRI monotherapy were categorized by genotype. C/C and C/G genotypes were classed together as “non-G/G.” The nominal, nonparametric data were assessed by Fisher's Exact Probability Test. The data indicate that individuals with a G/G genotype are significantly more likely to be taking a non-SSRI as opposed to an SSRI.

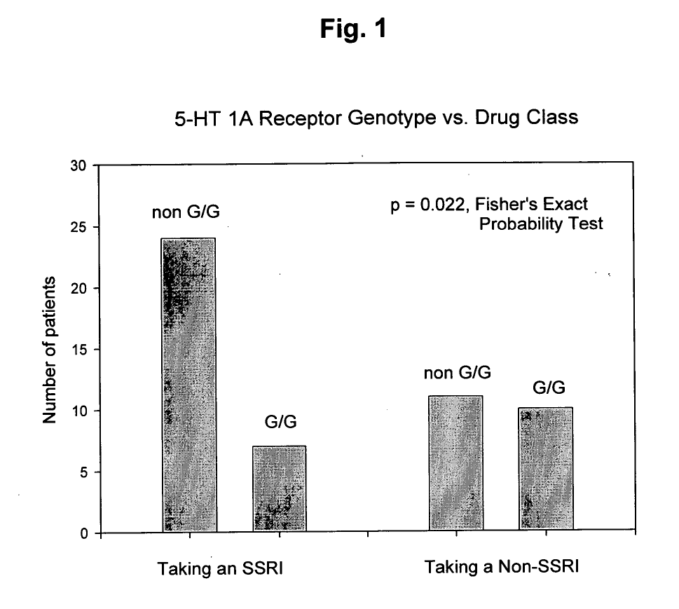

FIG. 2 is a bar graph depicting the relationship between 5-HT1A receptor genotype individuals and improvement on the PHQ9 (Patient Health Questionnaire) following treatment with an SSRI. Patients with moderately severe or severe depression (PHQ> or =15) were classed by genotype and their improvement on the PHQ9 was ranked. The ordinal, nonparametric data was analyzed by the Mann-Whitney U Test. Patients with the G/G genotype showed significantly less improvement.

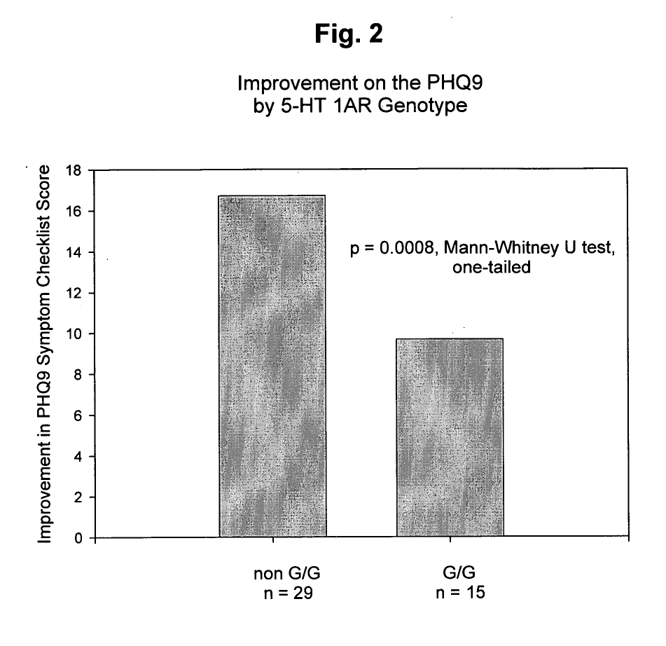

FIG. 3 is a line graph illustrating dependence of therapeutic response by 5-HT1A receptor G/G on norepinephrine (NE) selectivity of the drug used for therapy. Patients with moderately severe or severe depression (PHQ9 score > or =15) were included. Patients' PHQ9 improvement scores (n=14) were correlated with the selectivity of their current antidepressant for 5-HT relative to NE (Ki NE/Ki 5-HT) using Pearson's product moment correlation (r). While the r value did not reach statistical significance, the negative slope of the line indicates that patients with a G/G genotype in the SNP of the 5-HT1A receptor may respond better to a drug with a relative NE selectivity.

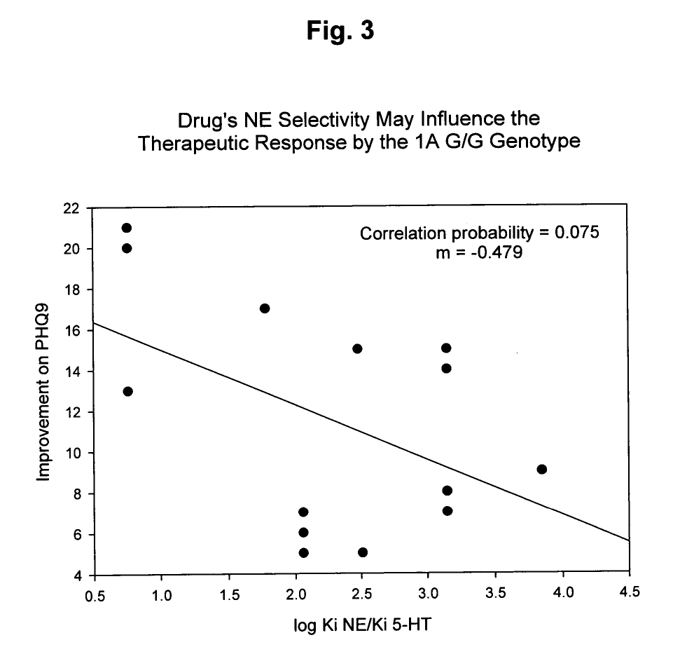

FIG. 4 is a bar graph illustrating the difference in improvement on the CGI (Clinical Global Impression Scale) between 5-HT1A receptor genotypes taking an SSRI. Patients with moderately severe or severe depression who were on either SSRI or non-SSRI monotherapy were included. Improvement on the CGI (clinician rated) was ranked by genotype: G/G vs. non-G/G (i.e., C/C and C/G). The ordinal, nonparametric data was evaluated by the Mann-Whitney U Test. Patients with the G/G genotype improved less than did other genotypes (p=0.02).

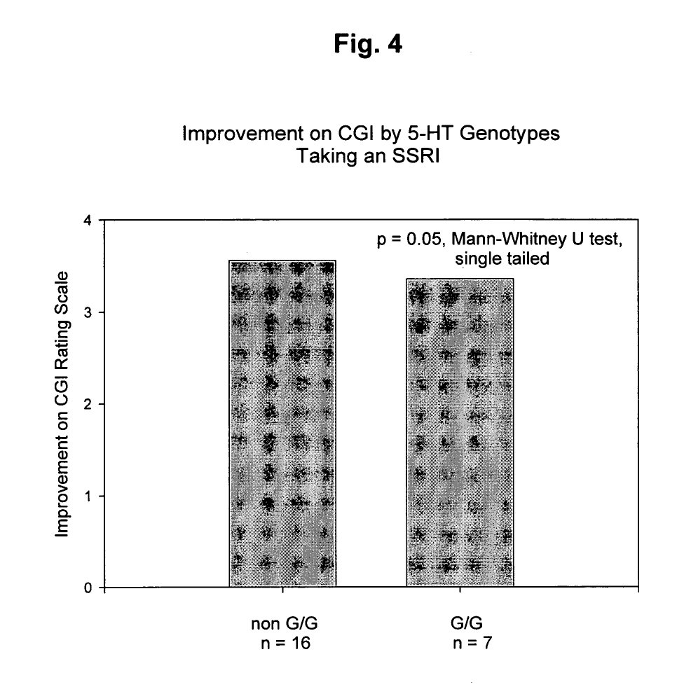

FIG. 5 is a bar graph illustrating the relationship between improvement on the CGI and 5-HT1A receptor genotype. Only patients with at least moderately severe depression who were on SSRI monotherapy were included. Improvement was ranked by genotype using the Mann-Whitney U test. Improvement by patients with the G/G genotype was less than that of other genotypes (p=0.05). There was no significant difference between genotypes for patients on non-SSRIs.

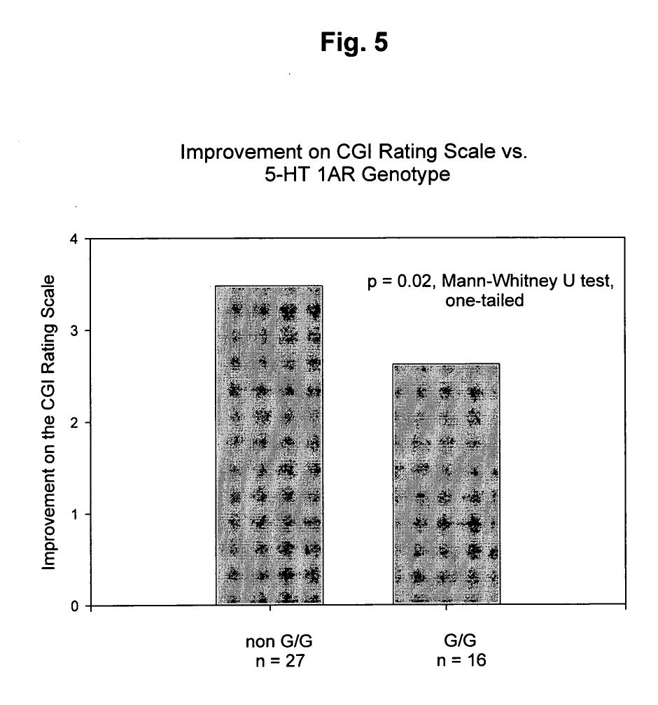

FIG. 6 is a bar graph illustrating current paroxetine usage by 5-HT2A receptor genotype. Only patients on SSRI monotherapy were included. Paroxetine use was compared to that of all other SSRIs by 5-HT2A genotype: non-C/C (i.e., T/T and T/C) vs. C/C. The incidence of paroxetine use by genotype was evaluated by Fisher's Exact Probability Test for nominal, non-parametric data. Although paroxetine use was dramatically lower in the patient group with the C/C genotype, the difference did not reach statistical significance.

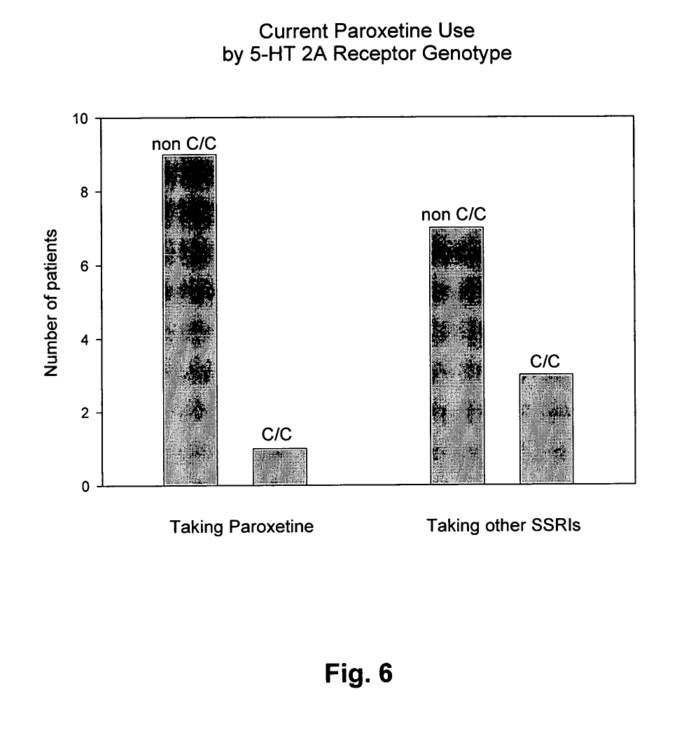

FIG. 7 is a bar graph illustrating the relationship between genotype and discontinuation of paroxetine and fluoxetine (another SSRI) due to side-effects. Incidence of discontinuation of the two SSRIs due to side effects was compared between genotypes. Fisher's Exact Probability Test was used to determine significance for the nominal data. While discontinuation of paroxetine was proportionally greater in patients with the C/C genotype, the difference was not statistically significant.

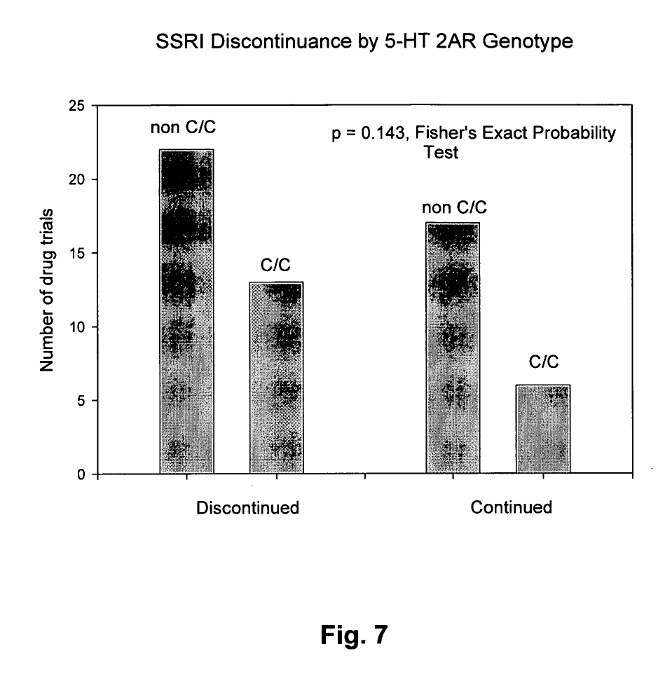

FIG. 8 is a graph illustrating the relationship between improvement on the PHQ9 by individuals having the 5-HT1A non-G/G genotype and the NE/5-HT selectivity of the drug used for therapy.

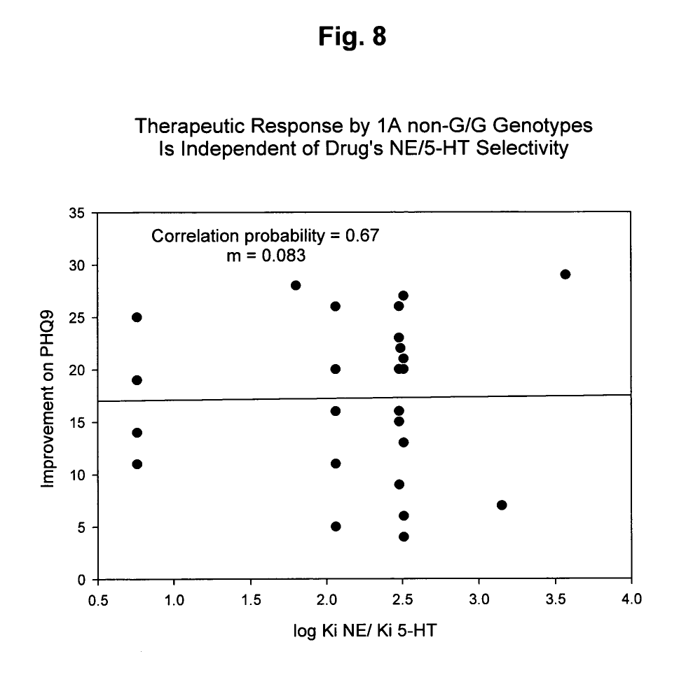

FIG. 9 is a graph illustrating the relationship between therapeutic response (as assessed by improvement on the PHQ9) by individuals having the 5-HT1A G/G genotype and the dopamine (DA) selectivity of the drug used for therapy.

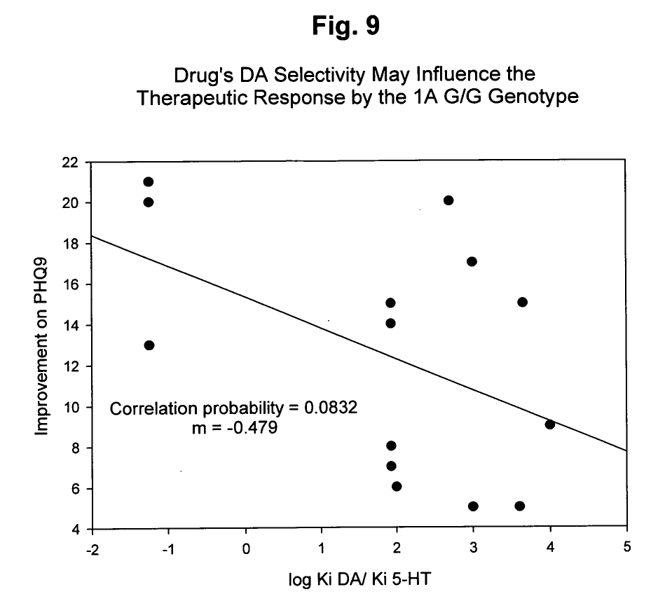

### DETAILED DESCRIPTION

The inventors have discovered that a genetic polymorphism (single nucleotide polymorphism, or SNP) at position −1019 of 5-HT_{1A}R is correlated with decreased effectiveness of SSRI antidepressant therapy. The G/G genotype correlates with decreased likelihood of improvement of symptoms of depression following SSRI antidepressant administration, as determined by both self-rating depression scale analysis and clinician-rated Clinical Global Impression rating.

The inventors have also discovered that a SNP at position 102 of the serotonin 2A receptor can be used to predict the likelihood that a patient will experience unwanted side effects if administered paroxetine or fluoxetine as antidepressant therapy, enabling a physician to better determine which medication to choose in order to improve response and decrease side effects of the therapy for a specific patient.

Inhibition of serotonergic raphe neurons is mediated by somatodendritic 5-HT_{1A} autoreceptors. Lemonde et al. reported an association between the C(−1019)G 5-HT_{1A}R promoter polymorphism and major depression. In depressed patients, the homozygous G(−1019) allele (G/G) was associated with a greater predisposition to depression and an even higher correlation with a predisposition for suicide than were other allelic variants at this position. (J. Neurosci. 2003, 23: 8788-8799.) One might therefore assume that individuals having this genotype would benefit from pharmacotherapeutics commonly prescribed to treat depression, including paroxetine and fluoxetine. Clinicians have noted, however, that not all individuals who take SSRIs for depression experience an improvement in their overall psychological health. In fact, many experience undesirable side-effects and must discontinue the prescribed treatment. Unfortunately, matching the individual with an effective medication has primarily been a matter of trial and error. The inventors have discovered that, although the G/G genotype is associated strongly with depression, individuals having this genotype do not respond as well to SSRI antidepressant therapy as do individuals who have been diagnosed with depression but who do not carry the G/G genotype at 5-HT1AR-1019. These individuals are commonly prescribed more than one antidepressant, often including both an SSRI and a non-SSRI.

The invention therefore provides a method for identifying individuals diagnosed with depression who will more likely to require alternate (e.g., non-SSRI) therapies to alleviate their symptoms—those individuals being less likely to benefit from conventional therapy such as, for example, administration of a single SSRI, when a patient is diagnosed with depression.

When the inventors compared individuals homozygous for the single nucleotide polymorphism in the serotonin 1A receptor promoter (G/G) to non-G/G genotypes with respect to type of anti-depressant monotherapy currently prescribed for those individuals, they found that seventy-percent (70%) of the individuals with the non-G/G genotype were using an SSRI whereas the usage of SSRIs for individuals with the G/G genotype was only 37.5%—about half that of individuals with the non-G/G genotype. When patients homozygous for G/G at −1019 in the serotonin receptor promoter were compared with other genotypes with respect to their self-reported improvement on the PHQ9, following administration of an SSRI to treat depression, the G/G genotype experienced only half the improvement of other genotypes.

Paroxetine hydrochloride (Paxil®, GlaxoSmithKline, Philadelphia, Pa.), or (−)-trans-4R-(4′-fluorophenyl)-3S-[(3′,4′-methylenedioxyphenoxy)methyl] piperidine hydrochloride hemihydrate, is a selective serotonin reuptake inhibitor (SSRI) that has been shown to block uptake of serotonin into human platelets. It is prescribed for a variety of depression- and anxiety-related disorders, with a recommended dosage of 10 to 20 mg administered daily.

When the inventors compared individuals homozygous for the single nucleotide polymorphism in the serotonin 2A receptor position 102, they discovered that the C/C homozygosity was generally associated with a low incidence of current paroxetine usage and a high rate of discontinuance of paroxetine due to side-effects. Only one out of ten individuals with the C/C genotype currently on an SSRI was taking paroxetine, as compared to nearly one-third of non-C/C individuals on SSRIs. The failure rate of for individuals with the C/C genotype taking paroxetine was almost two times greater than that for non-C/C individuals (40% vs. 75%), whereas the failure rates for SSRIs in general were comparable for the two groups (53% and 62%, respectively).

The inventors therefore provide a method for choosing treatment options for specific patients based upon identification of each patient's genotype at position −1019 of the serotonin 1A receptor gene and at position 102 of the serotonin 2A receptor. By identifying an individual as either −1019 G/G or non-G/G, a physician can determine whether a patient will be more or less likely to benefit from SSRI therapy without additional pharmaceutical intervention. Patients with the G/G genotype would generally be better served to be directed to either a non-SSRI medication or a combination therapy that does not rely on SSRIs alone. In choosing the appropriate SSRI, a physician can use the patient's genotype at position 102 of the serotonin 2A receptor to predict the likelihood that the patient will benefit from either paroxetine or fluoxetine.

Briefly, detecting the polymorphism of interest may be carried out by collecting a biological sample containing DNA from the subject, and then determining the presence or absence of the specific polymorphic sequence using DNA sequencing or other means known to those of skill in the art. Any biological sample that contains the DNA of that subject may be employed, including tissue samples and blood samples, although generally blood samples provide a more convenient source of subject DNA. The nucleotide sequence of 5-HT_{1A}R and its promoter is known (see GenBank accession numbers AJ781317, Z11168, NM000524, and NT006713 for example), as it the sequence of 5HT_{2A}R (see GenBank accession numbers NM000621, BC079576, BC074849, BC074848, AF498982). Appropriate probes, restriction enzyme digestion techniques, and other means of detecting a polymorphism such as the G/G polymorphism at position −1019 of 5-HT_{1A}R are known to those of skill in the art. Determining the presence or absence of a polymorphism in a DNA sample from an individual human subject may be carried out with a labeled oligonucleotide probe or similar means known to those of skill in the art of genetic analysis. Isolated DNA from the sample may first be amplified by polymerase chain reaction or ligase chain reaction.

Amplification of a target nucleic acid sequence may be carried out by a number of different means known to those of skill in the art. Examples of amplification techniques include, but are not limited to, polymerase chain reaction, ligase chain reaction, strand displacement amplification (Walker, G. et al., Proc. Natl. Acad. Sci. USA 89, 392-396 (1992); Walker, G. et al., Nucleic Acids Res. 20, 1691-1696 (1992)), transcription-based amplification (Kwoh, D. et al., Proc. Natl. Acad. Sci. USA 86, 1173-1177 (1989)), self-sustained sequence replication (or “3SR”) (Guatelli et al., Proc. Natl. Acad. Sci. USA 87, 1874-1878 (1990)), the Q.beta. replicase system (Lizardi, P. et al., BioTechnology 6, 1197-1202 (1988)), nucleic acid sequence-based amplification (NASBA) (Lewis, R. Genetic Engineering News 12 (9), 1 (1992)), repair chain reaction (RCR), and boomerang DNA amplification (BDA). Amplification techniques such as these may utilize probes that specifically bind to DNA or RNA comprising the polymorphism of interest (target sequence) but do not bind under the same hybridization conditions if the target sequence is absent. Probes and primers, including those for either amplification and/or protection, are comprised of nucleotides, or nucleotide bases (known DNA nucleotides such as adenine, guanine, cytosine, and thymine, and synthetic and/or modified nucleotides). Oligonucleotides or polynucleotides used as probes or primers are any suitable length, but are typically from about 5 to about 60 nucleotides in length, for example. Such probes and or primers may be immobilized on or coupled to a solid support such as a bead or chip by means described in the scientific literature and known to those of skill in the art, and/or coupled to or labeled with a detectable group such as a fluorescent compound, a chemiluminescent compound, a radioactive element or composition, or an enzyme. In the method of the present invention, the inventors used a primer pair comprising a first primer comprising the sequence 5′-GCTGGACTGTTAGATGATAACGGAGGTAC-3′ (SEQ ID NO: 1) and a second prier having the sequence 5′-TGTCAGCATCCCAGAGTGGCAATAGGAG-3′ (SEQ ID NO: 2) to amplify and sequence subject DNA to identify the −1019 polymorphism in the 5HT_{1A}R promoter. The inventors used a primer pair comprising a first primer comprising the sequence 5′-CTACAAGTTCTGGCTTAGACATGG-3′ (SEQ ID NO: 3) and a second primer comprising either 5′-CGATTTTCAGAGTCGACTGTCCAG-3′ (SEQ ID NO: 4) (for amplification), 5′-CGATTTTCAGAGTCGACTGT-3′ (SEQ ID NO: 5) (for cycle sequencing), or 5′-CGACGGTGAGAGGCACCCTCC-3′ (for both amplification and sequencing) to amplify and sequence subject DNA to identify the 102 polymorphism in the 5HT_{2A} gene sequence.

Polymerase chain reaction (PCR) may be carried out according to known techniques and protocols, such as those described in U.S. Pat. Nos. 4,683,195; 4,683,202; 4,800,159; and 4,965,188. Commercial kits are available to make the PCR procedure relatively easy to perform and to standardize. Ligase chain reaction (LCR) can also be performed by known techniques, such as that described by Weiss (Science 254, 1292 (1991)).

Kits useful for carrying out the methods of the present invention generally comprise one or more appropriate oligonucleotide probes or primers for hybridizing to the target sequence having the polymorphism to be detected, as well as appropriate reagents such as buffers and enzymes for carrying out the method of assaying for the polymorphism. In one embodiment, for example, a kit may comprise a set of primer pairs such as SEQ ID NO: 1 and SEQ ID NO: 2, in combination with appropriate reagents for amplification (e.g., Taq polymerase, PCR buffers, etc.) and sequencing the target DNA to detect the sequence at position −1019 of 5-HT1A. A kit may also comprise one set of primer pairs to evaluate the sample for the presence or absence of the G/G polymorphism at −1019 in the 5-HT1A and a second set of primer pairs (e.g., SEQ ID NO: 3 in combination with SEQ ID NO: 4 and SEQ ID NO: 5, or in combination with SEQ ID NO: 6) to evaluate the sample for the presence or absence of the C/C polymorphism at 102 in 5-HT2A. Kits may also comprise additional reagents for DNA isolation from human tissue, such as blood, epithelial cells obtained from the cheek, etc.

EasySNP® (Tecan Group Ltd., Maennedorf, Switzerland), for example, evaluates SNPs of interest that are amplified from genomic DNA using PCR, followed by degradation of excess primers and dNTPs. Procedures for genotyping of single nucleotide polymorphisms have also been described, for example, by Kinoshita-Kikuta et al. (Nuc. Acids Res. (2002) 30: e126), Waterfall and Cobb (Nuc. Acids Res. (2001) 29: e119). Detection of single nucleotide polymorphisms can be performed using acridinium labeled probes, as well. Briefly, an exact complimentary hybrid base protects the acridinium label by intercalation between the bases, while a single base mismatch at the site of the label exposes the acridinium ester to the solution of dilute base. Hydrolysis in dilute base for 30 minutes destroys chemiluminescence of the unhybridized and single mismatch probes, leaving a significant percentage of the exact match label intact. Therefore, matched hybrids luminesce, while unmatched hybrids do not.

Primers can also be used to sequence target regions of DNA in which a known polymorphism exists, such as in the present invention. Both the sequences surrounding (both 5′ to and 3′ to the specific nucleotide position where the target polymorphism exists) the 5-HT_{1A}R −1019 region and the 5-HT_{2A}R 102region are known. Appropriate primers can therefore be synthesized by those of skill in the art using known methods. Primers can be chosen based upon the known sequences upstream and downstream from the polymorphism site and can be synthesized by commercial laboratories so that they are readily available to those of skill in the art of DNA amplification and sequencing. Specific allelic combinations can be detected based upon the results of the sequencing reaction.

The invention can be further described by means of the following non-limiting examples:

### EXAMPLES

Patients were recruited for the study based upon diagnosis of major depression, administration of anti-depressant medication for a minimum of 6 weeks before the study was initiated, lack of administration of other psychotropic medication, and freedom from serious medical or psychiatric co-morbidity (e.g., unstable medical condition, drug or alcohol abuse, psychosis, serious psychiatric condition other than depression and/or anxiety). Blood was obtained by finger prick and preparation of patient (subject) DNA was performed using the BloodDirect™ PCR Buffer Kit. A one millimeter punch of blood sample from filter paper was suspended in Blood Direct™ PCR Buffer (Novagen) according to manufacturer's directions, along with dNTPs and Taq polymerase in a total volume of 50 μl. DNA was amplified in an Eppendorf Mastercycler gradient PCR for 38 cycles following initial denaturation at 80° C. for 15 minutes. The annealing step was performed for one minute at 63-64° C. for the 5HT1A receptor amplimer. Sequencing of target regions to detect the base pairs present at position −1019 of 5-HT_{1A}R and position 102 of 5-HT_{2A}R was performed in the Core Molecular Biology Facility of the East Tennessee State University College of Medicine, Johnson City, Tenn. Fifty-seven (57) subjects were enrolled in the study, although blood was not obtained from one subject. Fifty-five individuals were white, 1 black, 1 Asian. Eleven were male, 46 were female. The subjects were 19-72 years of age, with an average age of 47.

Subject medications were recorded, and antidepressants were classified as either serotonin reuptake inhibitors (SSRIs) or all other non-SSRI medications (non-SSRI). Subjects were categorized as either receiving SSRI or receiving a non-SSRI either in combination with an SSRI or alone. Fewer G/G patients received monotherapy with an SSRI alone than did non-G/G patients, indicating that SSRIs were not, when administered without additional pharmacotherapeutic agents, effective for decreasing symptoms of depression in G/G patients.

At intake, patients were assessed by clinicians, using the Clinical Global Impression (CGI, 7 pt. scale) system. Three ratings were given: present severity, past severity, and global improvement. Demographic data (age, gender, race), were obtained, along with psychiatric and drug treatment history, history of side effects (chronicity, severity, relationship to drug administered and type of action taken), non-drug psychiatric history. Patients were also asked to complete the PHQ9 nine symptom checklist for each time period comprising the last two weeks, the worse two weeks, and the best two weeks, with each item having a 0-3 scale as described by Kroenke (Kroenke, K. and Spitzer, R., Psychiatric Annals 52: 509-515, 2002). Improvement of symptoms was determined by subtracting the prior two weeks score from the worst weeks score.

Subjects who discontinued the prescribed SSRI due to unwanted side effects were assigned a positive score, while subjects who continued their SSRI therapy were assigned a negative score. Scores were then compared as a function of genotype at 5-HT_{2A}R position 109. The percentage of subjects that discontinued specific SSRI therapy due to side effects was assessed as a function of 5-HT_{2A}R102genotype. Subjects were scored as above, with discontinuance receiving a positive score and maintenance receiving a negative score.

Genotype at −1019 of the 5HT 1A receptor promoter was correlated with the type of antidepressant therapy being received. The G/G genotype was contrasted with other genotypes (G/C & C/C). Antidepressants were classed as either serotonin reuptake inhibitors (SSRIs) or all other antidepressants (non-SSRI). Patients were categorized as either receiving an SSRI alone or as receiving either a non-SSRI or an SSRI in combination with a non-SSRI (both-neither). Nominal nonparametric data were assessed by Fisher's Exact Probability Test. Results indicated that individuals having the G/G genotype are significantly more likely to be maintained on a non-SSRI, as opposed to an SSRI, than are non-GG individuals. When results from the study group were tabulated, more than three times as many individuals who had the non-G/G genotype were taking an SSRI as were G/G genotype individuals. About two times more non-G/G individuals were using SSRIs than were G/G individuals.

Comparative improvement was assessed following administration of SSRI therapy as determined by the PHQ9 (5HT1AR) self-rating depression scale as a function of genotype at −1019 of the serotonin 1A receptor. Patients with moderately severe or severe depression (PHQ9≧15) were classed by genotype and their improvement on the PHQ9 was ranked. The genotype “GG” was compared to other genotypes (C/G & C/C; “non-GG”). Improvement was obtained by subtracting the score for the “prior two weeks” from the score for the “worst two weeks”. The ordinal, nonparametric data was analyzed by the Mann-Whitney U test. Patients with G/G genotype showed significantly less improvement when treated with SSRI therapy.

Comparative improvement was assessed following administration of SSRI therapy as determined by the clinician-rated Clinical Global Impression (CGI) rating scale as a function of genotype at −1019 of the serotonin 1A receptor. Only patients with at least moderately severe depression who were on SSRI monotherapy were included in the analysis. The genotype “GG” was compared to other genotypes (C/G & C/C; “non-GG”). Improvement was ranked by genotype using the Mann-Whitney U test. Improvement for patients with the G/G genotype was at least one point less overall than that of other genotypes.

The percentage of patients in the study group who discontinued SSRI use due to side effects as a function of genotype at 102 of the serotonin 2A receptor was assessed. The genotype “C/C” was compared to other genotypes (C/T & T/T; “non-CC”). Each patient who had ever taken an SSRI was scored as either positive or negative for side effects. The reporting of drug discontinuance associated with a patient report of side effects to any SSRI was scored as positive. The difference between genotypes was not statistically significant.

Numbers relative to incidence of patient reports of discontinuance of an SSRI due to side effects as a function of genotype at 102 of the serotonin 2A receptor were evaluated. The genotype “C/C” was compared to other genotypes (C/T & T/T; “non-CC”). Each patient who had ever taken an SSRI was scored as either positive or negative for side effects. The reporting of side effects to any SSRI was score as positive. The difference between genotypes was not statistically significant. P=0.32. More than four times as many C/C genotype individuals in the study were taking non-paroxetine SSRIs than were non-C/C individuals, and at least twice as many individuals who are non-C/C took a non-paroxetine SSRI than did C/C individuals.

The percentage of patients discontinuing fluoxetine due to side effects as a function of genotype at 102 of the serotonin 2A receptor was determined. The genotype “C/C” was compared to other genotypes (C/T & T/T; “non-CC”). Each patient who had taken paroxetine was scored as either positive or negative for discontinuance associated with side effects. Data were analysed by Fisher's Exact Probability Test. P=0.03

The percentage of patients discontinuing paroxetine due to side effects as a function of genotype at 102 of the serotonin 2A receptor was determined. The genotype “C/C” was compared to other genotypes (C/T & T/T; “non-CC”). Each patient who had taken paroxetine was scored as either positive or negative for side effects.

What is claimed is:

1. A method for screening a patient to determine whether or not that patient is likely to benefit from therapeutic administration of a selective serotonin reuptake inhibitor for treatment of depression, the method comprising identifying the genotype of the individual at position −1019 in the DNA sequence of the 5-HT1A receptor gene, with the presence of the G/G genotype indicating that selective serotonin reuptake inhibitor treatment will be less likely to result in successful treatment of symptoms and the presence of a non-G/G genotype at the same position indicating that SSRI treatment will be more likely to result in successful treatment of symptoms.

2. A method for screening individuals who are more likely to experience unwanted side-effects related to administration of a selective serotonin reuptake inhibitor for the treatment of depression, the method comprising identifying the genotype of the individual at position 102 in the DNA sequence of the 5-HT2A receptor gene, with the presence of the C/C genotype indicating that selective serotonin reuptake inhibitor treatment will be more likely to result in unwanted side-effects and the presence of a non-C/C genotype at the same position indicating that SSRI treatment will be less likely to result in unwanted side-effects.

3. The method of claim 2 wherein the selective serotonin reuptake inhibitor is paroxetine.

4. A kit for pre-screening one or more patients prior to choosing a specific antidepressant therapy, the kit comprising a primer pair comprising SEQ ID NO: 1 and SEQ ID NO: 2.

5. The kit of claim 4 further comprising reagents for isolating DNA from the one or more patients.

6. The kit of claim 4 further comprising reagents for amplification of DNA from the one or more patients.

7. The kit of claim 4 further comprising SEQ ID NO: 3, SEQ ID NO: 4, and SEQ ID NO: 5.

8. The kit of claim 4 further comprising SEQ ID NO: 3 and SEQ ID NO: 6.
